(19) United States
(12) Patent Application Publication
(10) Pub. No.: US 2019/0314252 A1
(43) Pub. Date: Oct. 17, 2019

(54) OIL-IN-WATER EMULSION COSMETIC FOR EYELASHES

(71) Applicant: TOKIWA CORPORATION, Nakatsugawa-shi (JP)

(72) Inventors: Yuko IWATANI, Kawaguchi-shi (JP); Yuka YAMAWAKI, Kawaguchi-shi (JP)

(73) Assignee: TOKIWA CORPORATION, Nakatsugawa-shi (JP)

(21) Appl. No.: 16/379,808

(22) Filed: Apr. 10, 2019

(30) Foreign Application Priority Data
Apr. 11, 2018 (JP) 2018-076351

(51) Int. Cl. A61K 8/06; A61K 8/81; A61K 8/73; A61K 8/86; A61K 8/19; A61K 8/92; A61Q 1/10

(52) U.S. Cl. CPC A61K 8/062; A61K 8/8176; A61K 8/8182; A61K 8/8129; A61K 8/731; A61K 2800/596; A61K 8/19; A61K 8/922; A61Q 1/10; A61K 2800/48; A61K 2800/43; A61K 8/86

(57) ABSTRACT

An oil-in-water emulsion cosmetic for eyelashes includes a water-soluble film forming agent, a water-soluble thickener, a powder, and a surfactant, in which the water-soluble thickener includes at least one selected from the group consisting of a cellulose-based thickener and a polysaccharide-based thickener, the powder includes carbon black, and the surfactant includes a first non-ionic surfactant having a hydrophilic-lipophilic balance of 10 to 17 and a second non-ionic surfactant having a hydrophilic-lipophilic balance of less than 8.

### CROSS-REFERENCE TO RELATED APPLICATIONS

This application claims the benefit of priority from Japanese Patent Application No. 2018-076351, filed on Apr. 11, 2018, the entire contents of which are incorporated herein by reference.

### TECHNICAL FIELD

The present disclosure relates to an oil-in-water emulsion cosmetic for eyelashes.

### BACKGROUND

Cosmetics for eyelashes such as mascara have a make-up effect of accentuating the impression of eyes by making the eyelashes thick and long. Recently, oil-in-water emulsion cosmetics for eyelashes that can be readily removed with warm water have been increasingly popular. In addition, in terms of the make-up effect, not only volume in eyelashes but also a curling effect including both an amount of curl-up of the eyelashes and the ability to maintain the curl-up is emphasized.

However, the oil-in-water emulsion cosmetics have a slow drying speed since water is a main ingredient, and eyelashes are weighed down before a cosmetic film was solidified, which makes it difficult to curl up the eyelashes. In addition, even if the eyelashes were curled up, the external phase readily absorbs moisture since the external phase is an aqueous ingredient, and the cosmetic film becomes heavy over time and thus the curling effect is not maintained.

Techniques for maintaining the curling effect are described in Japanese Unexamined Patent Publication No. 2009-227605 and Japanese Unexamined Patent Publication No. 2005-112770 which are directed to a wax or a film forming agent that is mixed to an oil-in-water emulsion cosmetic for eyelashes. Japanese Unexamined Patent Publication No. 2005-298497 describes that a film forming agent having a specific film forming property and a weight-average molecular weight is mixed to a cosmetic for eyelashes including a volatile ingredient. Furthermore, Japanese Unexamined Patent Publication No. 2006-111536 describes that a hydrophobized porous silica is mixed to a cosmetic for eyelashes having an aqueous medium as a continuous phase.

### SUMMARY

Consumers may attempt to obtain a particular curling effect or volume by repetitively applying a cosmetic. In order to obtain sufficient volume, consumers may expect that clumps rarely occur even when the cosmetic is repetitively applied, a uniform cosmetic film is readily formed, and the cosmetic does not slide off the surface of the eyelashes, so that an appropriate amount of the cosmetic can remain attached to the eyelashes.

The techniques described in Japanese Unexamined Patent Publication No. 2009-227605, Japanese Unexamined Patent Publication No. 2005-112770, Japanese Unexamined Patent Publication No. 2005-298497, and Japanese Unexamined Patent Publication No. 2006-111536 are not adequate to simultaneously obtain the curling effect and volume at the high level expected by consumers. For example, the cosmetics of Japanese Unexamined Patent Publication No. 2009-227605, Japanese Unexamined Patent Publication No. 2005-112770, and Japanese Unexamined Patent Publication No. 2005-298497 include a wax or a film forming agent that do not have a sufficient curling effect, and do not sufficiently eliminate clumps or reducing sliding. In addition, since the wax or the film forming agent has an opaque appearance in many cases, a gloss of the cosmetic film is lost or appears to become white, and thus a gloss effect of the cosmetic that makes eyelashes look glossy is not obtained. In Japanese Unexamined Patent Publication No. 2006-111536, where a large quantity of powders is mixed to impart a curl-maintaining property to the cosmetic, there is a tendency that clumps readily occur and it is difficult to make a cosmetic film with no clumps.

Disclosed herein is an oil-in-water emulsion cosmetic for eyelashes that reduces the occurrence of clumps even when the cosmetic is repetitively applied, is excellent in adherence, and is capable of imparting gloss, a high curling effect, and volume to eyelashes.

In some examples, the an oil-in-water emulsion cosmetic for eyelashes may comprise (A) water-soluble film forming agent, (B) water-soluble thickener, (C) powder, and (D) surfactant. In some examples, the (B) water-soluble thickener comprises at least one selected from the group consisting of a cellulose-based thickener and a polysaccharide-based thickener, the (C) powder comprises carbon black, and the (D) surfactant comprises (D1) non-ionic surfactant having a hydrophilic-lipophilic balance (HLB) of 10 to 17 and (D2) non-ionic surfactant having an HLB less than 8.

Even when the oil-in-water emulsion cosmetic is repetitively applied, the oil-in-water emulsion cosmetic may be manufactured not to cause clumps, to be excellent in adherence, and to impart gloss, a high curling effect, and volume to eyelashes.

A carbon black having a specific gravity lighter than that of black iron oxide, may be used with a water-soluble film forming agent to facilitate a curl-up effect and a curl-maintaining power. By combining and mixing the carbon black with the above-mentioned ingredients, clumps do not occur even when repetitively applied, an appropriate amount of the cosmetic is applied to the eyelashes, and an appropriate amount of cosmetic film with no clumps is provided, thereby resulting in both a curling effect and volume at the same time while imparting gloss to the eyelashes.

The oil-in-water emulsion cosmetic for eyelashes may be used in mascara and the like since the carbon black is combined and mixed with the above ingredients so that a cosmetic film excellent in blackness may be formed.

In some examples, a content of the (C) powder is 0.01% by mass to 10.0% by mass based on the total amount of the oil-in-water emulsion cosmetic for eyelashes. For example, the contents may be selected or modified to affect certain characteristics of the cosmetic from a viewpoint of no occurrence of clumps and a curling effect of a cosmetic film.

From a viewpoint of no occurrence of clumps, a curling effect, gloss, and blackness of the cosmetic film, a content of the carbon black may be 0.01% by mass to 5.0% by mass based on the total amount of the oil-in-water emulsion cosmetic for eyelashes.

In addition, from a viewpoint of no occurrence of clumps and the curling effect of the cosmetic film, a content of the powder other than the carbon black may be equal to or less than 6.0% by mass based on the total amount of the oil-in-water emulsion cosmetic for eyelashes.

In some examples, a mass ratio (D1)/(D2) between (D1) non-ionic surfactant having an HLB of 10 to 17 and (D2) non-ionic surfactant having an HLB less than 8 is 1.4 to 6.0. By making a mixture ratio of a non-ionic surfactant having an HLB of 10 to 17 greater than that of a non-ionic surfactant having an HLB less than 8, fine emulsified particles are formed, a cosmetic film having gloss and blackness is obtained, and stability of the cosmetic is improved. In such an oil-in-water emulsion cosmetic for eyelashes, consistency is maintained, and thus repetitive application is readily adjusted and a desired make-up effect is readily obtained over a long period of time.

In some examples, from a viewpoint of stability and forming a cosmetic film having gloss, and blackness, a content of (D1) non-ionic surfactant having an HLB of 10 to 17 is 1.0% by mass to 10.0% by mass based on the total amount of the oil-in-water emulsion cosmetic for eyelashes, and a content of (D2) non-ionic surfactant having an HLB less than 8 is 0.5% by mass to 8.0% by mass based on the total amount of the oil-in-water emulsion cosmetic for eyelashes.

Accordingly, example oil-in-water emulsion cosmetics for eyelashes that do not cause clumps even when repetitively applied, is excellent in adherence, and can impart gloss, a high curling effect, and volume to eyelashes, are disclosed herein.

The oil-in-water emulsion cosmetics for eyelashes can also form a cosmetic film excellent in blackness. In addition, the oil-in-water emulsion cosmetics for eyelashes can have sufficient stability.

### DETAILED DESCRIPTION

Oil-in-Water Emulsion Cosmetic for Eyelashes

An example oil-in-water emulsion cosmetic for eyelashes may comprise (A) water-soluble film forming agent (referred to as “Ingredient (A)” in some cases), (B) water soluble thickener (referred to as “Ingredient (B)” in some cases), (C) powder (referred to as “Ingredient (C)” in some cases), and (D) surfactant (referred to as “Ingredient (D)” in some cases).

(A) Water-Soluble Film Forming Agent

Example water-soluble film forming agents include polyvinylpyrrolidone (PVP), polyvinylalcohol (PVA), and vinylpyrrolidone/vinyl acetate copolymer (VP/VA copolymer). One kind of Ingredient (A) can be used alone or two or more kinds thereof can be used in combination.

A content of Ingredient (A) in the oil-in-water emulsion cosmetic for eyelashes may be 0.01% by mass to 10% by mass, or 0.1% by mass to 5.0% by mass, based on the total amount of the oil-in-water emulsion cosmetic for eyelashes, from a viewpoint of usability, and readily imparting a curling effect and volume to eyelashes.

The oil-in-water emulsion cosmetic for eyelashes may comprise a water-insoluble film forming agent. Examples of the water-insoluble film forming agent include an alkyl acrylate copolymer, an alkyl acrylate polymer, an alkyl methacrylate copolymer, an alkyl methacrylate polymer, an alkyl acrylate/alkyl methacrylate copolymer, a vinyl acetate polymer, an alkyl acrylate/styrene copolymer, an alkyl methacrylate/styrene copolymer, a vinyl pyrrolidone/styrene copolymer, an organopolysiloxane polymer, and the like, or any combination thereof.

(B) Water-Soluble Thickener

Example water-soluble thickeners include a cellulose-based thickener, a polysaccharide thickener, a clay mineral, a starch-based thickener, an alginic acid-based thickener, an animal-based thickener. One kind of Ingredient (B) can be used alone or two or more kinds thereof can be used in combination.

In order to avoid the occurrence of clumps even when the cosmetic is repetitively applied and to obtain sufficient volume, Ingredient (B) may comprise at least one kind of water-soluble thickener (referred to as “Ingredient (B1)” in some cases) selected from the group of thickeners consisting of a cellulose-based thickener and a polysaccharide thickener.

Example cellulose-based thickeners include methyl cellulose, ethyl cellulose, carboxymethyl cellulose, hydroxyethyl cellulose, hydroxypropyl cellulose, hydroxypropyl methyl cellulose, and the like, or any combination thereof.

Example polysaccharide-based thickeners include pectin, guar gum, xanthan gum, carrageenan, gellan gum, gum Arabic, and the like, or any combination thereof.

The water-soluble thickener can impart thixotropic properties to a cosmetic for eyelashes. A cosmetic for eyelashes having thixotropic properties does not show fluidity in a state having no stress. However, since the cosmetic is subjected to shear while being applied to the eyelashes, its viscosity is lowered so that the cosmetic can have fluidity. Thus, a large amount of the cosmetic may be prevented from adhering to the surface all at once. The addition of Ingredient (A) may result in the reduction or elimination of the occurrence of clumps and sliding, and may provide for the repetitive application (or repetitive coating) of the cosmetic and a uniform cosmetic film.

A content of Ingredient (B) in the oil-in-water emulsion cosmetic for eyelashes may be 0.01% by mass to 1% by mass, or 0.01% by mass to 0.5% by mass based on the total amount of the oil-in-water emulsion cosmetic for eyelashes, from a viewpoint of avoiding the occurrence of clumps even when the cosmetic is repetitively applied and readily imparting volume.

(C) Powder

A cosmetic powder may be mixed in the oil-in-water emulsion cosmetic for eyelashes, and example powders include a constitution powder, a coloring pigment, and the like. The powder may vary in terms of: a shape such as spherical shape, plate shape, and needle shape; a particle diameter such as haze shape, fine particle, and pigment class; and a particle structure such as porous structure and imperforate structure. One kind of Ingredient (C) can be used alone or two or more kinds thereof can be used in combination.

Ingredient (C) may comprise carbon black. By combining carbon black with Ingredient (B1), a higher curl-maintaining power can be obtained and a cosmetic film excellent in gloss and blackness may be formed.

From a viewpoint of the curling effect, carbon black having a specific gravity of 1.5 to 2.5 g/cm³ can be used. The carbon black may include a commercially available product such as DK BLACK No. 2 (manufactured by Daito Kasei Co., Ltd., specific gravity: 1.7 to 1.9 g/cm³).

A content of the carbon black in the oil-in-water emulsion cosmetic for eyelashes may be 0.01% by mass to 5.0% by mass, 0.1% by mass to 4.0% by mass, or 0.1% by mass to 2.0% by mass based on the total amount of the oil-in-water emulsion cosmetic for eyelashes, from a viewpoint of forming a cosmetic film having a curling effect, gloss, and blackness.

In addition, the content of the carbon black in the oil-in-water emulsion cosmetic for eyelashes may be equal to or less than 10.0% by mass based on the total amount of the oil-in-water emulsion cosmetic for eyelashes, from a viewpoint of preventing the cosmetic from being excessively hardened due to oil adsorption properties of the carbon black and preventing the formation of clumps.

Ingredient (C), other than carbon black, may comprise an organic coloring pigment such as black iron oxide, Bengala (red iron oxide), yellow iron oxide, cobalt oxide, chromium oxide, ultramarine blue, dark blue, titanium oxide, and zinc oxide; an organic coloring pigment such as Red No. 228, Red No. 226, Blue No. 404, Red No. 202, Yellow No. 4 aluminum lake; a pearl pigment such as mica titanium, fish scale guanine, and bismuth oxychloride; a natural dye such as carmine and safflower; a constitution pigment such as glass powder, silicic anhydride, aluminum silicate, magnesium silicate, aluminum magnesium silicate, mica, synthetic mica, synthetic sericite, sericite, talc, kaoline, silicon carbide, and barium sulfate; or a spherical powder such as crosslinked polymethylmethacrylate and silicone elastomer, or any combination thereof.

In addition, the oil-in-water emulsion cosmetic for eyelashes may comprise a fiber, as Ingredient (C) other than carbon black. Examples of the fiber include a nylon fiber, a rayon fiber, a polypropylene fiber, a polyester fiber, a cellulose fiber, or any combination thereof. A length of the fiber may be 0.1 to 10 mm, or 0.3 to 7 mm. A dimension of the fiber may be 0.1 to 25 TeX (hereinafter, simply indicated as “T”), or 0.3 to 20 T. In some examples, one kind or two or more kinds of the fibers having different shapes, dimensions and/or lengths can be used.

A content of Ingredient (C) in the oil-in-water emulsion cosmetic for eyelashes may be 0.01% by mass to 10.0% by mass based on the total amount of the oil-in-water emulsion cosmetic for eyelashes, from a viewpoint of imparting no occurrence of clumps, a curling effect, gloss, and blackness to the cosmetic film.

A content of the powder other than carbon black may be equal to or less than 6.0% by mass, or equal to or less than 4.0% by mass based on the total amount of the oil-in-water emulsion cosmetic for eyelashes, from a viewpoint of no occurrence of clumps and the curling effect of the cosmetic film.

(D) Surfactant

A cosmetic surfactant may be mixed in the oil-in-water emulsion cosmetic for eyelashes. In some examples, two or more of Ingredient (D) can be combined for use as the surfactant.

The oil-in-water emulsion cosmetic for eyelashes may comprise (D1) non-ionic surfactant having an HLB of 10 to 17 (referred to as “Ingredient (D1)” in some cases) and (D2) non-ionic surfactant having an HLB less than 8 (referred to as “Ingredient (D2)” in some cases), as Ingredient (D), from a viewpoint of forming a cosmetic film having stability, gloss, and blackness. One kind of each of Ingredient (D1) and Ingredient (D2) can be used alone, or two or more kinds thereof can be used in combination.

Examples of the non-ionic surfactant having an HLB of 10 to 17 include polyoxyethylene hydrogenated castor oil, polyoxyethylene sorbitan fatty acid ester, and the like, or any combination thereof. From a viewpoint of stability, polyoxyethylene hydrogenated castor oil may be used.

Examples of the non-ionic surfactant having an HLB less than 8 include glycerin fatty acid ester, sorbitan fatty acid ester, self-emulsifying glycerin fatty acid ester, and the like, or any combination thereof.

A content of Ingredient (D1) in the oil-in-water emulsion cosmetic for eyelashes may be 1.0% by mass to 10.0% by mass, 2.5% by mass to 8.0% by mass, or 4.2% by mass to 8.0% by mass based on the total amount of the oil-in-water emulsion cosmetic for eyelashes, from a viewpoint of forming a cosmetic film having stability, gloss, and blackness.

A content of Ingredient (D2) in the oil-in-water emulsion cosmetic for eyelashes may be 0.5% by mass to 8.0% by mass, 0.5% by mass to 3.0% by mass, or 0.5% by mass to 2.0%/o by mass based on the total amount of the oil-in-water emulsion cosmetic for eyelashes, from a viewpoint of forming a cosmetic film having stability, gloss, and blackness.

A mass ratio (D1)/(D2) between Ingredient (D1) and Ingredient (D2) may be 1.4 to 6.0, 1.8 to 6.0, or 3.3 to 6.0. The mass ratio (D)/(D2) may be set to such a range to form a fine emulsified particle, to obtain a cosmetic film having gloss and blackness, and to improve stability of the cosmetic.

The oil-in-water emulsion cosmetic for eyelashes may comprise a surfactant such as a non-ionic surfactant other than Ingredient (D1) and Ingredient (D2), an anionic surfactant, a cationic surfactant, and an ampholytic surfactant. One kind of the surfactant can be used alone, or two or more kinds thereof can be used in combination.

Examples of the anionic surfactant include a higher fatty acid, a higher fatty acid salt containing a higher fatty acid and alkali, and the like, or any combination thereof. The higher fatty acid may be stearic acid and palmitic acid, from a viewpoint of emulsification stability. The alkali may be triethanolamine, 2-amino-2-methyl-1-propanol (AMP), and 2-amino-2-methyl-1,3-propandiol (AMPD), from a viewpoint of emulsification stability.

The oil-in-water, emulsion cosmetic for eyelashes may comprise a higher fatty acid salt, from a viewpoint of improving adherence and dispersibility of carbon black. A content of the higher fatty acid salt may be 0.5% by mass to 10.0% by mass, or 1.0% by mass to 6.0% by mass based on the total amount of the oil-in-water emulsion cosmetic for eyelashes.

Other Ingredients

The oil-in-water emulsion cosmetic for eyelashes may comprise one or more aqueous ingredients and one or more oily ingredients other than the above-described ingredients.

The aqueous ingredient includes a water phase (external phase) of the oil-in-water emulsion cosmetic for eyelashes, and examples thereof include water, a polyvalent alcohol such as 1,3-butylene glycol, dipropylene glycol, glycerin, 1,2-pentanediol, sorbitol, and mannitol; and a lower alcohol such as ethanol, propyl alcohol, isopropyl alcohol, and isobutyl alcohol. One kind of these can be used alone, or two or more kinds thereof can be used in combination.

The oily ingredient includes an oil phase (internal phase) of the oil-in-water emulsion cosmetic for eyelashes, and examples thereof include fat and oil, waxes, a carbohydrate, an ester oil, a higher alcohol, a higher fatty acid, a silicone oil, and an ultraviolet absorber. One kind of these can be used alone, or two or more kinds thereof can be used in combination.

Examples of the fat and oil include solid fat and oil such as a hydrogenated castor oil, a hydrogenated jojoba oil, a pearl oil, a vegetable wax, and liquid fat and oil such as a jojoba seed oil, a sunflower seed oil, an olive oil, a castor oil, a macadamia nut oil, a camellia oil, a rapeseed oil, a linseed oil, triglycerin, and the like, or any combination thereof.

Examples of the waxes include a beeswax, a carnauba wax, a candelilla wax, a rice bran wax, a sunflower seed wax, a rice wax, a whale wax, a lanolin wax, and the like, or any combination thereof.

Examples of the carbohydrate include a polyethylene wax, a microcrystalline wax, a Fisher Tropsch wax, ceresin, a petroleum jelly, liquid paraffin, squalane, a mineral oil, and the like, or any combination thereof.

Examples of the ester oil include glyceryl tribehenate, a cholesterol fatty acid ester, diisostearyl malate, isopropyl myristate, 2-ethyl hexanoic acid cetyl, isopropyl palmitate, ethylhexyl palmitate, tricapryl/capric acid glyceryl, dicapric acid neopentylglycol, tri-2-ethyl hexanoic acid glyceryl, triisostearic acid polyglyceryl, tetra (hydroxystearic acid/isostearic acid) dipentaerithrityl, di-2-ethylhexanoic acid neopentylglycol, triethylhexanoin, a dimer acid ester, and the like, or any combination thereof.

Examples of the higher alcohol include stearyl alcohol, behenyl alcohol, cetyl alcohol, cetostearyl alcohol, oleyl alcohol, octyldodecanol, isostearyl alcohol, and the like, or any combination thereof.

Examples of the higher fatty acid include stearic acid, oleic acid, myristic acid, palmitic acid, isostearic acid, behenic acid, linolic acid, linoleic acid, and the like, or any combination thereof.

Examples of the silicone oil include dimethylpolysiloxane, methylphenylpolysiloxane, octamethyl cyclotetrasiloxane, decamethyl cyclopentasiloxane, methyl trimethicone, and the like, or any combination thereof.

The oil-in-water emulsion cosmetic for eyelashes may comprise other cosmetic ingredients such as a humectant, an ultraviolet absorber, a preservative, vitamins, a cosmetic ingredient, an antioxidant, and a fragrance. They can be blended with or otherwise added to one or more of the above-mentioned ingredients.

In some examples, the consistency of the oil-in-water emulsion cosmetic for eyelashes may be 0.05 to 0.8, or 0.1 to 0.5, from a viewpoint of usability. The consistency of the cosmetic may be measured by the following method.

Measurement of Consistency

A cosmetic melted by heating was filled in a 30 mL screw cup, and a sample for measurement was left overnight at 25° C. In the sample for measurement, by using a FUDOH rheometer RT-2002 D·D (manufactured by Rheotec Co., Ltd.), the consistency of the cosmetic was measured at 25° C., with a pressure sensitive axis of 8ϕ spherical shape, at a needle penetration rate of 6 cm/min, and at a needle penetration depth of 10 mm.

Examples of the use of the oil-in-water emulsion cosmetic for eyelashes include a mascara, a mascara foundation, a mascara overcoat, and the like.

Production Method of the Oil-in-Water Emulsion Cosmetic for Eyelashes

An example production method of the oil-in-water emulsion cosmetic for eyelashes may comprise preparing, mixing and emulsifying the above-described water phase portion including Ingredient (A), Ingredient (B) and other aqueous ingredients thereof, Ingredient (C), and the above-described oil phase portion including Ingredient (D) and the oily ingredients.

### Additional Examples

Hereinafter, more details of the oil-in-water emulsion cosmetic for eyelashes are described with reference to examples. Numerical values in tables indicate contents (% by mass) based on the total amount of the cosmetic base.

Example evaluation methods of the oil-in-water emulsion cosmetics are described below.

Evaluation of Sense of Use and Impression of Eyes

Twenty panels specialized in cosmetic evaluation were applied with the example oil-in-water emulsion cosmetics and the comparative examples about 10 times, and a five-stage evaluation was performed on the following evaluation items according to the following evaluation criteria to give evaluation points to each of samples. Then, averages of the evaluation points of all panels were determined according to the following determination criteria.

Evaluation Items

(a) No occurrence of clumps: evaluation on whether or not clumps occur when the cosmetic is repetitively applied

(b) Adherence: evaluation on whether or not an appropriate amount of the cosmetic adheres to eyelashes without sliding

(c) Gloss: evaluation on whether or not gloss is present in the cosmetic film

(d) Blackness: evaluation on whether or not blackness is present in the cosmetic film

(e) Curling effect: evaluation on whether or not eyelashes are curled up after use of the cosmetic, and on whether or not after that, the curl is maintained after 6 hours of the normal life of the panel

### Evaluation Points: Evaluation Criteria

5 points: Extremely good

4 points: Good

3 points: Normal

2 points: Poor

1 point: Extremely poor

### Determination Criteria (Average Points of Evaluation Points)

A: Equal to or more than 4

B: Equal to or more than 3 and less than 4

C: Equal to or more than 2 and less than 3

D: Less than 2

In a case where evaluation on no occurrence of clumps and adherence of the cosmetic film is favorable, usability is excellent, the adjustment of the volume is facilitated, and an evaluation on gloss, blackness, and the curling effect is favorable, the overall make-up effect of the cosmetic such as an impression of the eyes is readily obtained.

(f) Stability

Regarding stability, based on a state immediately after preparation of each sample, each sample was stored in a thermostatic bath of 50° C. for 1 month, and changes in consistency and presence or absence of a discharge liquid were evaluated according to the following four-stage criteria.

### Determination Criteria

A: No change

B: Slight change

C: Some change

D: Marked change

### Examples 1 to 14 and Comparative Examples 1 to 7

Example oil-in-water emulsion cosmetics for eyelashes having each of the compositions shown in Tables 1 to 5 were prepared by the following production method, and the above evaluation thereon was performed. The results are shown in Tables 1 to 5.

Production Method

Ingredients 1 to 13 were mixed together, heated to 90° C., and melted, and uniformly mixed to produce an oil phase. Subsequently, Ingredients 14 to 28 which were heated to 90° C. and melted were added to the oil phase, and emulsified. The emulsified product was cooled, and then added with Ingredient 29 and stirred. The resultant product was filled in a container, and an oil-in-water emulsion cosmetic for eyelashes was obtained.

TABLE 1

| | | Example 1 | Example 2 | Example 3 |
|---|---|---|---|---|
| 1 | Carnauba wax | 6 | 6 | 6 |
| 2 | Beeswax | 6 | 6 | 6 |
| 3 | Stearic acid | 2 | 2 | 2 |
| 4 | Palmitic acid | 1 | 1 | 1 |
| 5 | Cetostearyl alcohol | 2 | 2 | 2 |
| 6 | Tocopherol | Appropriate amount | Appropriate amount | Appropriate amount |
| 7 | Simethicone | Appropriate amount | Appropriate amount | Appropriate amount |
| 8 | Polyoxyethylene hydrogenated castor oil | 5.5 | 5.2 | — |
| | (HLB 15) | | | |
| 9 | Monooleic acid polyoxyethylene sorbitan | — | — | 5.0 |
| | (20E.O.) (HLB 15) | | | |
| 10 | Polyoxyethylene hydrogenated castor oil | — | — | — |
| | (HLB 12.5) | | | |
| 11 | Monostearic acid polyethylene glycol | — | — | — |
| | (HLB 17.5) | | | |
| 12 | Lipophilic monostearic acid glycerin | 1 | — | 1.5 |
| | (HLB 3) | | | |
| 13 | Monostearic acid sorbitan (HLB 4.7) | — | 1.3 | — |
| 14 | Purified water | Balance | Balance | Balance |
| 15 | 1,3-Butylene glycol | 5 | 5 | 5 |
| 16 | Preservative | Appropriate amount | Appropriate amount | Appropriate amount |
| 17 | Triethanolamine | 1 | 1 | 1 |
| 18 | Polyvinyl pyrrolidone *1 | 1 | 1 | 1 |
| 19 | Polyvinyl alcohol *2 | — | — | — |
| 20 | Vinyl acetate/vinyl pyrrolidone | — | — | — |
| | copolymer *3 | | | |
| 21 | Xanthan gum | 0.3 | 0.3 | 0.3 |
| 22 | Sodium carboxymethyl cellulose | — | — | — |
| 23 | Hydroxyethyl cellulose | — | — | — |
| 24 | Hydroxypropyl cellulose | — | — | — |
| 25 | Hydroxypropyl methyl cellulose | — | — | — |
| 26 | Gum Arabic | — | — | — |
| 27 | Carbon black *4 | 1 | 1 | 1 |
| 28 | Black iron oxide *5 | — | — | — |
| 29 | Alkyl acrylate copolymer emulsion | 30 | 30 | 30 |
| | (solid content 45%) *6 | | | |
| (a) No occurrence of clumps | | A | A | B |
| (b) Adherence | | A | B | A |
| (c) Gloss | | A | A | A |
| (d) Blackness | | A | A | A |
| (e) Curling effect | | A | A | A |
| (f) Stability | | A | A | B |
| | | Example 4 | Example 5 | Example 6 |
| 1 | Carnauba wax | 6 | 6 | 6 |
| 2 | Beeswax | 6 | 6 | 6 |
| 3 | Stearic acid | 2 | 2 | 2 |
| 4 | Palmitic acid | 2 | 1 | 1 |
| 5 | Cetostearyl alcohol | 2 | 2 | 2 |
| 6 | Tocopherol | Appropriate amount | Appropriate amount | Appropriate amount |
| 7 | Simethicone | Appropriate amount | Appropriate amount | Appropriate amount |
| 8 | Polyoxyethylene hydrogenated castor oil | — | 5.5 | 5.5 |
| | (HLB 15) | | | |
| 9 | Monooleic acid polyoxyethylene sorbitan | — | — | — |
| | (20E.O.) (HLB 15) | | | |
| 10 | Polyoxyethylene hydrogenated castor oil | 5.5 | — | — |
| | (HLB 12.5) | | | |
| 11 | Monostearic acid polyethylene glycol | — | — | — |
| | (HLB 17.5) | | | |
| 12 | Lipophilic monostearic acid glycerin | 1 | 1 | 1 |
| | (HLB 3) | | | |
| 13 | Monostearic acid sorbitan (HLB 4.7) | — | — | — |
| 14 | Purified water | Balance | Balance | Balance |
| 15 | 1,3-Butylene glycol | 5 | 5 | 5 |
| 16 | Preservative | Appropriate amount | Appropriate amount | Appropriate amount |
| 17 | Triethanolamine | 1 | 1 | 1 |
| 18 | Polyvinyl pyrrolidone *1 | 1 | — | — |
| 19 | Polyvinyl alcohol *2 | — | 1 | — |
| 20 | Vinyl acetate/vinyl pyrrolidone | — | — | 1 |
| | copolymer *3 | | | |
| 21 | Xanthan gum | 0.3 | 0.3 | 0.3 |
| 22 | Sodium carboxymethyl cellulose | — | — | — |
| 23 | Hydroxyethyl cellulose | — | — | — |
| 24 | Hydroxypropyl cellulose | — | — | — |
| 25 | Hydroxypropyl methyl cellulose | — | — | — |
| 26 | Gum Arabic | — | — | — |
| 27 | Carbon black *4 | 1 | 1 | 1 |
| 28 | Black iron oxide *5 | — | — | — |
| 29 | Alkyl acrylate copolymer emulsion | 30 | 30 | 30 |
| | (solid content 45%) *6 | | | |

| | | | |
|---|---|---|---|
| (a) No occurrence of clumps | A | A | A |
| (b) Adherence | A | A | B |
| (c) Gloss | A | A | A |
| (d) Blackness | A | A | A |
| (e) Curling effect | A | A | A |
| (f) Stability | B | A | A |

TABLE 2

| | | Example 7 | Example 8 | Example 9 |
|---|---|---|---|---|
| 1 | Carnauba wax | 6 | 6 | 6 |
| 2 | Beeswax | 6 | 6 | 6 |
| 3 | Stearic acid | 2 | 2 | 2 |
| 4 | Palmitic acid | 1 | 1 | 1 |
| 5 | Cetostearyl alcohol | 2 | 2 | 2 |
| 6 | Tocopherol | Appropriate amount | Appropriate amount | Appropriate amount |
| 7 | Simethicone | Appropriate amount | Appropriate amount | Appropriate amount |
| 8 | Polyoxyethylene hydrogenated castor oil | 5.5 | 5.5 | 5.5 |
| | (HLB 15) | | | |
| 9 | Monooleic acid polyoxyethylene sorbitan | — | — | — |
| | (20E.O.) (HLB 15) | | | |
| 10 | Polyoxyethylene hydrogenated castor oil | — | — | — |
| | (HLB 12.5) | | | |
| 11 | Monostearic acid polyethylene glycol | — | — | — |
| | (HLB 17.5) | | | |
| 12 | Lipophilic monostearic acid glycerin | 1 | 1 | 1 |
| | (HLB 3) | | | |
| 13 | Monostearic acid sorbitan (HLB 4.7) | — | — | — |
| 14 | Purified water | Balance | Balance | Balance |
| 15 | 1,3-Butylene glycol | 5 | 5 | 5 |
| 16 | Preservative | Appropriate amount | Appropriate amount | Appropriate amount |
| 17 | Triethanolamine | 1 | 1 | 1 |
| 18 | Polyvinyl pyrrolidone *1 | 1 | 1 | 1 |
| 19 | Polyvinyl alcohol *2 | — | — | — |
| 20 | Vinyl acetate/vinyl pyrrolidone | — | — | — |
| | copolymer *3 | | | |
| 21 | Xanthan gum | — | — | — |
| 22 | Sodium carboxymethyl cellulose | 0.3 | — | — |
| 23 | Hydroxyethyl cellulose | — | 0.3 | — |
| 24 | Hydroxypropyl cellulose | — | — | 0.3 |
| 25 | Hydroxypropyl methyl cellulose | — | — | — |
| 26 | Gum Arabic | — | — | — |
| 27 | Carbon black *4 | 1 | 1 | 1 |
| 28 | Black iron oxide *5 | — | — | — |
| 29 | Alkyl acrylate copolymer emulsion | 30 | 30 | 30 |
| | (solid content 45%) *6 | | | |

| | | | |
|---|---|---|---|
| (a) No occurrence of clumps | A | B | B |
| (b) Adherence | B | A | A |
| (c) Gloss | A | A | A |
| (d) Blackness | A | A | A |
| (e) Curling effect | A | A | A |
| (f) Stability | A | A | A |

| | | Example 10 | Example 11 |
|---|---|---|---|
| 1 | Carnauba wax | 6 | 6 |
| 2 | Beeswax | 6 | 6 |
| 3 | Stearic acid | 2 | 2 |
| 4 | Palmitic acid | 1 | 1 |
| 5 | Cetostearyl alcohol | 2 | 2 |
| 6 | Tocopherol | Appropriate amount | Appropriate amount |
| 7 | Simethicone | Appropriate amount | Appropriate amount |
| 8 | Polyoxyethylene hydrogenated castor oil | 5.5 | 5.5 |
| | (HLB 15) | | |
| 9 | Monooleic acid polyoxyethylene sorbitan | — | — |
| | (20E.O.) (HLB 15) | | |
| 10 | Polyoxyethylene hydrogenated castor oil | — | — |
| | (HLB 12.5) | | |
| 11 | Monostearic acid polyethylene glycol | — | — |
| | (HLB 17.5) | | |
| 12 | Lipophilic monostearic acid glycerin | 1 | 1 |
| | (HLB 3) | | |
| 13 | Monostearic acid sorbitan (HLB 4.7) | — | — |
| 14 | Purified water | Balance | Balance |
| 15 | 1,3-Butylene glycol | 5 | 5 |
| 16 | Preservative | Appropriate amount | Appropriate amount |
| 17 | Triethanolamine | 1 | 1 |
| 18 | Polyvinyl pyrrolidone *1 | 1 | 0.5 |
| 19 | Polyvinyl alcohol *2 | — | — |
| 20 | Vinyl acetate/vinyl pyrrolidone | — | — |
| | copolymer *3 | | |
| 21 | Xanthan gum | — | — |
| 22 | Sodium carboxymethyl cellulose | — | — |
| 23 | Hydroxyethyl cellulose | — | — |
| 24 | Hydroxypropyl cellulose | — | — |
| 25 | Hydroxypropyl methyl cellulose | 0.3 | — |
| 26 | Gum Arabic | — | 0.5 |
| 27 | Carbon black *4 | 1 | 1 |
| 28 | Black iron oxide *5 | — | — |
| 29 | Alkyl acrylate copolymer emulsion | 30 | 30 |
| | (solid content 45%) *6 | | |

| | | |
|---|---|---|
| (a) No occurrence of clumps | A | B |
| (b) Adherence | A | A |
| (c) Gloss | A | A |
| (d) Blackness | A | A |
| (e) Curling effect | A | A |
| (f) Stability | B | B |

TABLE 3

| | | Example 12 | Example 13 | Example 14 |
|---|---|---|---|---|
| 1 | Carnauba wax | 6 | 6 | 6 |
| 2 | Beeswax | 6 | 6 | 6 |
| 3 | Stearic acid | 2 | 2 | 2 |
| 4 | Palmitic acid | 1 | 1 | 1 |
| 5 | Cetostearyl alcohol | 2 | 2 | 2 |
| 6 | Tocopherol | Appropriate amount | Appropriate amount | Appropriate amount |
| 7 | Simethicone | Appropriate amount | Appropriate amount | Appropriate amount |
| 8 | Polyoxyethylene hydrogenated castor oil | 4.2 | 3.8 | 9.0 |
| | (HLB 15) | | | |
| 9 | Monooleic acid polyoxyethylene sorbitan | — | — | — |
| | (20E.O.) (HLB 15) | | | |
| 10 | Polyoxyethylene hydrogenated castor oil | — | — | — |
| | (HLB 12.5) | | | |
| 11 | Monostearic acid polyethylene glycol | — | — | — |
| | (HLB 17.5) | | | |
| 12 | Lipophilic monostearic acid glycerin | 2.3 | 2.7 | 2.0 |
| | (HLB 3) | | | |
| 13 | Monostearic acid sorbitan (HLB 4.7) | — | — | — |
| 14 | Purified water | Balance | Balance | Balance |
| 15 | 1,3-Butylene glycol | 5 | 5 | 5 |
| 16 | Preservative | Appropriate amount | Appropriate amount | Appropriate amount |
| 17 | Triethanolamine | 1 | 1 | 1 |
| 18 | Polyvinyl pyrrolidone *1 | 1 | 1 | 1 |
| 19 | Polyvinyl alcohol *2 | — | — | — |
| 20 | Vinyl acetate/vinyl pyrrolidone | — | — | — |
| | copolymer *3 | | | |
| 21 | Xanthan gum | 0.3 | 0.3 | 0.3 |
| 22 | Sodium carboxymethyl cellulose | — | — | — |
| 23 | Hydroxyethyl cellulose | — | — | — |
| 24 | Hydroxypropyl cellulose | — | — | — |
| 25 | Hydroxypropyl methyl cellulose | — | — | — |
| 26 | Gum Arabic | — | — | — |
| 27 | Carbon black *4 | 1 | 1 | 1 |
| 28 | Black iron oxide *5 | — | — | — |
| 29 | Alkyl acrylate copolymer emulsion | 30 | 30 | 30 |
| | (solid content 45%) *6 | | | |

| | | | |
|---|---|---|---|
| (a) No occurrence of clumps | A | A | B |
| (b) Adherence | A | A | A |
| (c) Gloss | B | B | A |
| (d) Blackness | B | B | A |
| (e) Curling effect | A | A | A |
| (f) Stability | A | A | B |

TABLE 4

| | | Comparative | Comparative | Comparative |
|---|---|---|---|---|
| | | Example 1 | Example 2 | Example 3 |
| 1 | Carnauba wax | 6 | 6 | 6 |
| 2 | Beeswax | 6 | 6 | 6 |
| 3 | Stearic acid | 2 | 2 | 2 |
| 4 | Palmitic acid | 1 | 1 | 1 |
| 5 | Cetostearyl alcohol | 2 | 2 | 2 |
| 6 | Tocopherol | Appropriate amount | Appropriate amount | Appropriate amount |
| 7 | Simethicone | Appropriate amount | Appropriate amount | Appropriate amount |
| 8 | Polyoxyethylene hydrogenated castor oil | 5.5 | 5.5 | 5.5 |
| | (HLB 15) | | | |
| 9 | Monooleic acid polyoxyethylene sorbitan | — | — | — |
| | (20E.O.) (HLB 15) | | | |
| 10 | Polyoxyethylene hydrogenated castor oil | — | — | — |
| | (HLB 12.5) | | | |
| 11 | Monostearic acid polyethylene glycol | — | — | — |
| | (HLB 17.5) | | | |
| 12 | Lipophilic monostearic acid glycerin | 1 | 1 | 1 |
| | (HLB 3) | | | |
| 13 | Monostearic acid sorbitan (HLB 4.7) | — | — | — |
| 14 | Purified water | Balance | Balance | Balance |
| 15 | 1,3-Butylene glycol | 5 | 5 | 5 |
| 16 | Preservative | Appropriate amount | Appropriate amount | Appropriate amount |
| 17 | Triethanolamine | 1 | 1 | 1 |
| 18 | Polyvinyl pyrrolidone *1 | — | — | 1 |
| 19 | Polyvinyl alcohol *2 | — | — | — |
| 20 | Vinyl acetate/vinyl pyrrolidone | — | — | — |
| | copolymer *3 | | | |
| 21 | Xanthan gum | — | 0.3 | — |
| 22 | Sodium carboxymethyl cellulose | — | — | — |
| 23 | Hydroxyethyl cellulose | — | — | — |
| 24 | Hydroxypropyl cellulose | — | — | — |
| 25 | Hydroxypropyl methyl cellulose | — | — | — |
| 26 | Gum Arabic | — | — | — |
| 27 | Carbon black *4 | 1 | 1 | 1 |
| 28 | Black iron oxide *5 | — | — | — |
| 29 | Alkyl acrylate copolymer emulsion | 30 | 30 | 30 |
| | (solid content 45%) *6 | | | |

| | | | |
|---|---|---|---|
| (a) No occurrence of clumps | C | B | D |
| (b) Adherence | D | C | C |
| (c) Gloss | A | A | A |
| (d) Blackness | A | A | A |
| (e) Curling effect | D | D | B |
| (f) Stability | A | A | A |

| | | Comparative | Comparative |
|---|---|---|---|
| | | Example 4 | Example 5 |
| 1 | Carnauba wax | 6 | 6 |
| 2 | Beeswax | 6 | 6 |
| 3 | Stearic acid | 2 | 2 |
| 4 | Palmitic acid | 2 | 1 |
| 5 | Cetostearyl alcohol | 2 | 2 |
| 6 | Tocopherol | Appropriate amount | Appropriate amount |
| 7 | Simethicone | Appropriate amount | Appropriate amount |
| 8 | Polyoxyethylene hydrogenated castor oil | 5.5 | 5.5 |
| | (HLB 15) | | |
| 9 | Monooleic acid polyoxyethylene sorbitan | — | — |
| | (20E.O.) (HLB 15) | | |
| 10 | Polyoxyethylene hydrogenated castor oil | — | — |
| | (HLB 12.5) | | |
| 11 | Monostearic acid polyethylene glycol | — | — |
| | (HLB 17.5) | | |
| 12 | Lipophilic monostearic acid glycerin | 1 | — |
| | (HLB 3) | | |
| 13 | Monostearic acid sorbitan (HLB 4.7) | — | — |
| 14 | Purified water | Balance | Balance |
| 15 | 1,3-Butylene glycol | 5 | 5 |
| 16 | Preservative | Appropriate amount | Appropriate amount |
| 17 | Triethanolamine | 1 | 1 |
| 18 | Polyvinyl pyrrolidone *1 | 1 | 1 |
| 19 | Polyvinyl alcohol *2 | — | — |
| 20 | Vinyl acetate/vinyl pyrrolidone | — | — |
| | copolymer *3 | | |
| 21 | Xanthan gum | 0.3 | 0.3 |
| 22 | Sodium carboxymethyl cellulose | — | — |
| 23 | Hydroxyethyl cellulose | — | — |
| 24 | Hydroxypropyl cellulose | — | — |
| 25 | Hydroxypropyl methyl cellulose | — | — |
| 26 | Gum Arabic | — | — |
| 27 | Carbon black *4 | — | 1 |
| 28 | Black iron oxide *5 | 8.5 | — |
| 29 | Alkyl acrylate copolymer emulsion | 30 | 30 |
| | (solid content 45%) *6 | | |

| | | |
|---|---|---|
| (a) No occurrence of clumps | C | C |
| (b) Adherence | B | C |
| (c) Gloss | D | D |
| (d) Blackness | D | D |
| (e) Curling effect | C | D |
| (f) Stability | B | D |

TABLE 5

| | | Comparative | Comparative |
|---|---|---|---|
| | | Example 6 | Example 7 |
| 1 | Carnauba wax | 6 | 6 |
| 2 | Beeswax | 6 | 6 |
| 3 | Stearic acid | 2 | 2 |
| 4 | Palmitic acid | 1 | 1 |
| 5 | Cetostearyl alcohol | 2 | 2 |
| 6 | Tocopherol | Appropriate amount | Appropriate amount |
| 7 | Simethicone | Appropriate amount | Appropriate amount |
| 8 | Polyoxyethylene hydrogenated castor oil | — | — |
| | (HLB 15) | | |
| 9 | Monooleic acid polyoxyethylene sorbitan | — | — |
| | (20E.O.) (HLB 15) | | |
| 10 | Polyoxyethylene hydrogenated castor oil | — | — |
| | (HLB 12.5) | | |
| 11 | Monostearic acid polyethylene glycol | — | 4.5 |
| | (HLB 17.5) | | |
| 12 | Lipophilic monostearic acid glycerin | 1.0 | 2 |
| | (HLB 3) | | |
| 13 | Monostearic acid sorbitan (HLB 4.7) | — | — |
| 14 | Purified water | Balance | Balance |
| 15 | 1,3-Butylene glycol | 5 | 5 |
| 16 | Preservative | Appropriate amount | Appropriate amount |
| 17 | Triethanolamine | 1 | 1 |
| 18 | Polyvinyl pyrrolidone *1 | 1 | 1 |
| 19 | Polyvinyl alcohol *2 | — | — |
| 20 | Vinyl acetate/vinyl pyrrolidone | — | — |
| | copolymer *3 | | |
| 21 | Xanthan gum | 0.3 | 0.3 |
| 22 | Sodium carboxymethyl cellulose | — | — |
| 23 | Hydroxyethyl cellulose | — | — |
| 24 | Hydroxypropyl cellulose | — | — |
| 25 | Hydroxypropyl methyl cellulose | — | — |
| 26 | Gum Arabic | — | — |
| 27 | Carbon black *4 | 1 | 1 |
| 28 | Black iron oxide *5 | — | — |
| 29 | Alkyl acrylate copolymer emulsion | 30 | 30 |
| | (solid content 45%) *6 | | |

| | | |
|---|---|---|
| (a) No occurrence of clumps | D | C |
| (b) Adherence | D | C |
| (c) Gloss | D | D |
| (d) Blackness | D | D |
| (e) Curling effect | D | B |
| (f) Stability | D | D |

In Tables 1 to 5, the details of footnotes “*1” to “*6” are as follows.

*1: PVP K-90 (manufactured by ISP Japan Ltd., product name)

*2: Gohsenol EG-25 (manufactured by Nippon Synthetic Chemical Industry Co., Ltd., product name)

*3: PVP/VA S-630 (manufactured by ISP Japan Ltd., product name)

*4: DK BLACK No. 2 (manufactured by Daito Kasei Co., Ltd., product name, specific gravity 1.8)

*5: TAROX BL-100 (manufactured by Titan Kogyo, Ltd., product name, specific gravity 5)

*6: Yodosol GH800F (manufactured by Akzo Nobel N.V., product name)

As shown in Tables 1 to 5, the cosmetics of Examples 1 to 14 were evaluated as “B” or higher in all the evaluation items of (a) no occurrence of clumps, (b) adherence, (c) gloss, (d) blackness, (e) curling effect, and (f) stability.

### Example 15: Mascara (Containing Fiber)

| (Ingredient) | (Mixture ratio (% by mass)) |
|---|---|
| 1. Carnauba wax | 4.0 |
| 2. Beeswax | 4.0 |
| 3. Stearic acid | 3.0 |
| 4. Stearyl alcohol | 2.0 |
| 5. Tocopherol | Appropriate amount |
| 6. Simethicone | Appropriate amount |
| 7. PEG-60 hydrogenated castor oil | 5.0 |
| 8. Stearic acid glyceryl | 1.5 |
| 9. Carbon black *4 | 1.0 |
| 10. Nylon fiber | 2.0 |
| 11. Purified water | Residual amount |
| 12. 1,3-butylene glycol | 5.0 |
| 13. Preservative | Appropriate amount |
| 14. AMP | 1.0 |
| 15. Polyvinyl pyrrolidone *1 | 1.0 |
| 16. Xanthan gum | 0.2 |
| 17. Alkyl acrylate copolymer emulsion *6 | 25.0 |

Details of footnotes “*1”, “*4” and “*6” are the same as those already described above.

Production Method

Ingredients (1) to (10) were heated to approximately 90° C., and uniformly mixed to be made into an oil phase. Subsequently, Ingredients (11) to (16) which were heated to approximately 90° C. were added to the obtained oil phase, and emulsified. The emulsified product was cooled, and then added to Ingredient (17) and stirred. The resultant product was filled in a container, and an oil-in-water emulsion mascara (containing a fiber) was obtained.

Evaluation

The obtained mascara (containing a fiber) was evaluated in the same manner as described above, and the mascara was evaluated as “A” in all the evaluation items of (a) no occurrence of clumps, (b) adherence, (c) gloss, (d) blackness, (e) curling effect, and (f) stability.

### Example 16: Mascara Foundation

| (Ingredient) | (Mixture ratio (% by mass)) |
|---|---|
| 1. Paraffin wax | 3.0 |
| 2. Beeswax | 5.0 |
| 3. Stearic acid | 2.0 |
| 4. Palmitic acid | 1.0 |
| 5. Cetostearyl alcohol | 1.0 |
| 6. Tocopherol | Appropriate amount |
| 7. Simethicone | Appropriate amount |
| 8. PEG-60 hydrogenated castor oil | 5.0 |
| 9. Stearic acid glyceryl | 1.5 |
| 10. Carbon black *4 | 0.05 |
| 11. Silica | 2.0 |
| 12. Purified water | Residual amount |
| 13. 1,3-Butylene glycol | 5.0 |
| 14. Preservative | Appropriate amount |
| 15. Triethanolamine | 1.0 |
| 16. Polyvinyl alcohol *2 | 1.0 |
| 17. Xanthan gum | 0.1 |
| 18. Alkyl acrylate copolymer emulsion *6 | 30.0 |

Details of footnotes “*2”, “*4” and “*6” are the same as those already described above.

Production Method

Ingredients (1) to (11) were heated to approximately 90° C., and uniformly mixed to be made into an oil phase. Subsequently, Ingredients (12) to (17) which were heated to approximately 90° C. were added to the obtained oil phase, and emulsified. The emulsified product was cooled, and then added to Ingredient (18) and stirred. The resultant product was filled in a container, and mascara foundation was obtained.

The obtained mascara foundation was evaluated in the same manner as described above, and the mascara foundation was evaluated as “A” in all the evaluation items of (a) no occurrence of clumps, (b) adherence, (c) gloss, (e) curling effect, and (f) stability.

It is to be understood that not all aspects, advantages and features described herein may necessarily be achieved by, or included in, any one particular example embodiment. Indeed, having described and illustrated various examples herein, it should be apparent that other examples may be modified in arrangement and detail. We claim all modifications and variations coming within the spirit and scope of the subject matter claimed herein.

What is claimed is:

1. An oil-in-water emulsion cosmetic for eyelashes, comprising:
(A) water-soluble film forming agent;
(B) water-soluble thickener,
(C) powder; and
(D) surfactant,
wherein the (B) water-soluble thickener comprises at least one selected from the group consisting of a cellulose-based thickener and a polysaccharide-based thickener,
wherein the (C) powder comprises carbon black, and
wherein the (D) surfactant comprises (D1) non-ionic surfactant having a hydrophilic-lipophilic balance (HLB) of 10 to 17 and (D2) non-ionic surfactant having an HLB less than 8.

2. The oil-in-water emulsion cosmetic according to claim 1,
wherein a content of the (C) powder is 0.01% by mass to 10.0% by mass based on a total amount of the oil-in-water emulsion cosmetic.

3. The oil-in-water emulsion cosmetic according to claim 1,
wherein a content of the carbon black is 0.01% by mass to 5.0% by mass based on a total amount of the oil-in-water emulsion cosmetic.

4. The oil-in-water emulsion cosmetic according to claim 1,
wherein a content of the powder other than the carbon black is equal to or less than 6.0% by mass based on a total amount of the oil-in-water emulsion cosmetic.

5. The oil-in-water emulsion cosmetic according to claim 1,
wherein a mass ratio (D1)/(D2) between the (D1) non-ionic surfactant having an HLB of 10 to 17 and the (D2) non-ionic surfactant having an HLB less than 8 is 1.4 to 6.0.

6. The oil-in-water emulsion cosmetic according to claim 1,
wherein a content of the (D1) non-ionic surfactant having an HLB of 10 to 17 is 1.0% by mass to 10.0% by mass based on a total amount of the oil-in-water emulsion cosmetic, and
wherein a content of the (D2) non-ionic surfactant having an HLB less than 8 is 0.5% by mass to 8.0% by mass based on the total amount of the oil-in-water emulsion cosmetic for eyelashes.

7. The oil-in-water emulsion cosmetic according to claim 1,
wherein a content of the (A) water-soluble film forming agent is 0.01% by mass to 10% by mass based on a total amount of the oil-in-water emulsion cosmetic.

8. The oil-in-water emulsion cosmetic according to claim 1,
wherein a content of the (B) water-soluble thickener is 0.01% by mass to 1% by mass based on a total amount of the oil-in-water emulsion cosmetic.

9. The oil-in-water emulsion cosmetic according to claim 1,
wherein (A) water-soluble film forming agent comprises at least one selected from the group consisting of polyvinylpyrrolidone (PVP), polyvinylalcohol (PVA), and vinylpyrrolidone/vinyl acetate copolymer (VP/VA copolymer).

10. The oil-in-water emulsion cosmetic according to claim 1,
wherein the cellulose-based thickener comprises at least one selected from the group consisting of methyl cellulose, ethyl cellulose, carboxymethyl cellulose, hydroxyethyl cellulose, hydroxypropyl cellulose, and hydroxypropyl methyl cellulose.

11. The oil-in-water emulsion cosmetic according to claim 1,
wherein the polysaccharide-based thickener comprises at least one selected from the group consisting of pectin, guar gum, xanthan gum, carrageenan, gellan gum, and gum Arabic.

12. The oil-in-water emulsion cosmetic according to claim 1,
wherein the (C) powder further comprises one or more fibers.

13. The oil-in-water emulsion cosmetic according to claim 1,
wherein the (D1) non-ionic surfactant having an HLB of 10 to 17 comprises at least one selected from the group consisting of polyoxyethylene hydrogenated castor oil and polyoxyethylene sorbitan fatty acid ester.

14. The oil-in-water emulsion cosmetic according to claim 1,
wherein the (D2) non-ionic surfactant having an HLB less than 8 comprises at least one selected from the group consisting of glycerin fatty acid ester, sorbitan fatty acid ester, and self-emulsifying glycerin fatty acid ester.

15. The oil-in-water emulsion cosmetic according to claim 1, further comprising one or more aqueous ingredients constituting a water phase of the oil-in-water emulsion cosmetic and one or more oily ingredients constituting an oil phase of the oil-in-water emulsion cosmetic.

16. The oil-in-water emulsion cosmetic according to claim 1,
wherein the oil-in-water emulsion cosmetic has a consistency of 0.05 to 0.8.

17. A method of providing a curling effect to eyelashes comprising:
applying an oil-in-water emulsion cosmetic to eyelashes,
the oil-in-water emulsion cosmetic comprising:
(A) water-soluble film forming agent;
(B) water-soluble thickener,
(C) powder; and
(D) surfactant,
wherein the (B) water-soluble thickener comprises at least one selected from the group consisting of a cellulose-based thickener and a polysaccharide-based thickener,
wherein the (C) powder comprises carbon black, and
wherein the (D) surfactant comprises (D1) non-ionic surfactant having an HLB of 10 to 17 and (D2) non-ionic surfactant having an HLB less than 8.

18. A method of manufacturing an oil-in-water emulsion cosmetic comprising:
preparing a water phase portion including a water-soluble film forming agent, a water-soluble thickener including at least one selected from the group consisting of a cellulose-based thickener and a polysaccharide-based thickener, and a powder including carbon black;
preparing an oil phase portion including a surfactant including a first non-ionic surfactant having a hydrophilic-lipophilic balance (HLB) of 10 to 17 and a second non-ionic surfactant having an HLB less than 8; and
combining the water phase portion and the oil phase portion to form the oil-in-water emulsion cosmetic.

19. The method according to claim 18,
wherein a content of the carbon black is 0.01% by mass to 5.0% by mass based on a total amount of the oil-in-water emulsion cosmetic.

20. The method according to claim 18,
wherein the powder further comprises one or more fibers.
